(19) United States
(12) Patent Application Publication
(10) Pub. No.: US 2026/0354855 A1
(43) Pub. Date: Nov. 12, 2026

(54) Endoscopic Instrument Reel

(71) Applicant: Modern Practical Innovations, LLC, Knoxville, TN (US)

(72) Inventors: Johnny Altawil, Knoxville, TN (US); Ammar Eli Al Tawil, Rochester Hills, MI (US)

(73) Assignee: Modern Practical Innovations, LLC, Knoxville, TN (US)

(21) Appl. No.: 19/201,508

(22) Filed: May 7, 2025

(51) Int. Cl. A61B 17/32; A61B 17/00; A61B 17/3205

(52) U.S. Cl. CPC A61B 17/320016; A61B 17/32056; A61B 2017/00367; A61B 2017/00438

(57) ABSTRACT

An endoscopic instrument reel having a thumb ring and finger rings for manipulation of operating wire relative to sheathing, and integrated into an enclosed reel for storing the sheathing/wire prior to deployment into the body of a patient and after withdrawal of the sheathing/wire from the body of the patient to avoid exposure of the sheathing/wire to the surrounding environment and medical personal.

### FIELD

This disclosure relates to the field of endoscopy. More particularly, the disclosure relates to an endoscopic instrument reel configured to enable storage and deployment of endoscopic sheathing and operating wire, and providing operative functionality of the operating wire to deploy, retrieve and operate tools for surgical procedures.

### BACKGROUND

Improvement is desired in the construction of endoscopic devices of the type having a flexible tubing or sheathing surrounding a flexible operating wire movable relative to the sheathing, and introduced into the body for operative purposes. One example involves polypectomy snares, although a variety of tools may be utilized at the distal end of the operating wire for different procedures.

Conventionally, the sheathing/wire structure is inserted into the body of a patient either through a scope or directly through a surgical incision or anatomical orifice, such as the rectum, and deployed to the site of interest using a rotatable deployment device. U.S. Pat. No. 5,066,295 entitled Rotatable Surgical Snare as seen in FIG. 1 is an example of one such prior art device.

The prior art device of FIG. 1 has a linear handle connected to a sheath housing an operating wire for retracting and protracting a snare at the end of the operating wire, and for connection to a source of electric power. The operating wire and sheath structure generally has a length ranging from about 4 feet to over 15 feet.

Snares can be utilized cold, to frictionally cut a polyp, or hot, to utilize electrical power to facilitate cutting of the polyp. Electrical power may also be applied to the operating wire to the wire for other purposes, such as tissue resection purposes, referred to as electro-resectioning.

The snare at the distal end of the operating wire is retracted into the sheathing until it is desired to expose the snare to collect a polyp, at which time the operating wire is manipulated to extend the snare shape out of the sheathing and surround the polyp. The operating wire is then manipulated to draw the snare back to the end of the sheathing to sever the polyp, with the sheathing/wire then being withdrawn from the body.

The sheathing/wire is exposed and extends from the handle as shown in FIG. 1. During the procedure, the operating wire is manipulated relative to the end of the sheathing by use of a thumb ring and finger rings. Upon retraction of the sheathing/wire from the body, the sheathing/wire remains exposed.

Deployments devices of the type shown in FIG. 1 having a handle with thumb and finger rings are in widespread use and medical practitioners develop experience in using such devices. However, such devices have shortcomings. For example, upon withdrawal of the sheathing/wire from the body, the sheathing/wire is covered with bodily fluids and the medical practitioners carrying out the procedure may undesirably come into contact with the sheathing/wire and the bodily fluids thereon.

Accordingly, what is desired is a device that retains a thumb ring and finger rings for manipulation/rotation of operating wire relative to sheathing, but which avoids the shortcomings of exposure of the sheathing/wire to the surrounding environment and medical personal as described.

The present disclosure advantageously provides an endoscopic instrument reel that has a thumb ring and finger rings for manipulation/rotation of operating wire relative to sheathing, and integrated into an enclosed reel for storing the sheathing/wire prior to deployment from the body of a patient and after withdrawal of the sheathing/wire from the body of the patient to avoid exposure of the sheathing/wire to the surrounding environment and medical personal.

The endoscopic instrument reel also has improved aesthetics as compared to conventional endoscopic devices with a unique appearance.

### SUMMARY

The above and other needs are met by an improved endoscopic instrument reel.

In one aspect, an endoscopic instrument reel according to the disclosure includes sheathing containing an operating wire slidably located within the sheathing, the operating wire having a proximal wire end extending from a proximal sheathing end of the sheathing and having a distal wire end that can exit and enter a distal sheathing end of the sheathing; a wheel assembly having a wire controlling wheel and a sheathing holding wheel assembled together to enable the wire controlling wheel to selectively rotate back and forth a limited amount relative to the sheathing holding wheel, the wheel assembly configured to receive the sheathing containing the operating wire, the wire controlling wheel having a channel, a first finger ring extending inwardly from the channel, and an operating wire receiver and locking pin located in the channel.

The sheathing holding wheel has a locking channel configured to cooperate with the channel of the wire controlling wheel and sized to receive the sheathing containing the operating wire and configured to fixedly engage the proximal sheathing end within the locking channel, a thumb ring extending inwardly of the locking channel, and a stop located in the locking channel configured to engage the locking pin of the wire controlling wheel and limit rotation of the wire holding wheel when the wire controlling wheel is rotated in a direction and amount to contact the locking pin with the stop. A housing encloses the wheel assembly and includes a passage for the sheathing containing the operating wire, the housing including a second finger ring extending in an outwardly direction on a side of the housing opposite of the first finger ring and the thumb ring;

A user may hold the endoscopic instrument reel with a first finger of the user in the first finger ring, a second finger of the user in the second finger ring, and a thumb of the user in the thumb ring, and the user may wind and unwind the sheathing containing the wire to and from the wheel assembly. The distal end of the operating wire may be selectively moved in and out of the distal end of the sheathing by selectively rotating the wire holding wheel relative to the sheathing holding wheel by use of the first finger ring, the second finger ring, and the thumb ring.

In another aspect, an endoscopic instrument reel according to the disclosure includes a sheathing containing an operating wire slidably located within the sheathing, the operating wire having a distal wire end that can exit and enter a distal sheathing end of the sheathing; a wire controlling wheel and a sheathing holding wheel assembled together to receive the sheathing containing the operating wire, the wire controlling wheel having a first finger ring, and the sheathing holding wheel having a thumb ring; and a housing operatively associated with the wire controlling wheel and the sheathing holding wheel, the housing having a passage for the sheathing containing the operating wire, the housing including a second finger ring.

A user may hold the endoscopic instrument reel with a first finger of the user in the first finger ring, a second finger of the user in the second finger ring, and a thumb of the user in the thumb ring, and the user may wind and unwind the sheathing containing the wire to and from the endoscopic instrument, and the distal end of the operating wire may be selectively moved in and out of the distal end of the sheathing by use of the first finger ring, the second finger ring, and the thumb ring.

### BRIEF DESCRIPTION OF THE DRAWINGS

Further advantages of the disclosure are apparent by reference to the detailed description when considered in conjunction with the figures, which are not to scale so as to more clearly show the details, wherein like reference numbers indicate like elements throughout the several views, and wherein:

FIG. 1 shows a prior art rotatable surgical snare.

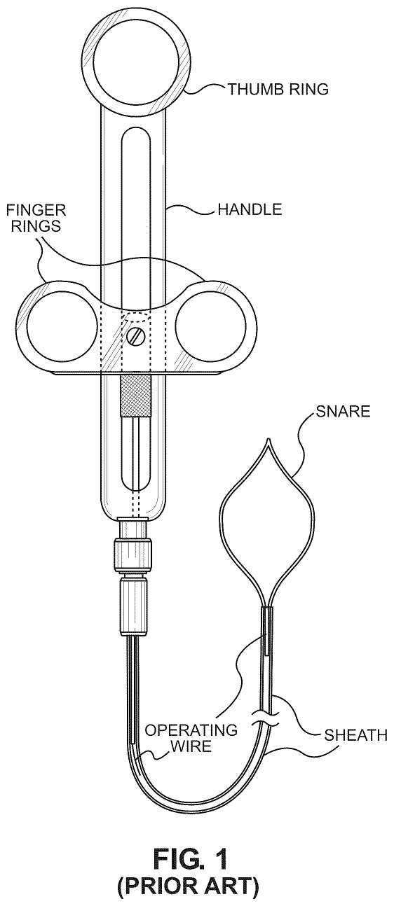

FIG. 2 is an upper perspective view of the endoscopic instrument reel.

FIG. 3 is an upper perspective view of the endoscopic instrument reel showing an operating wire extending from sheathing.

FIG. 4 is a lower perspective view of the endoscopic instrument reel.

FIG. 5 is an exploded perspective view of the endoscopic instrument reel.

FIG. 6 is an outer view of a top cover of the endoscopic instrument reel.

FIG. 7 is an inner view of the top cover.

FIG. 8 is an outer view of a wire controlling wheel of the endoscopic instrument reel.

FIG. 9 is an inner view of the wire controlling wheel.

FIG. 10 is an inner view of a tube holding wheel of the endoscopic instrument reel.

FIG. 11 is an outer view of the tube holding wheel.

FIG. 12 is an inner view of a bottom cover of the endoscopic instrument reel.

FIG. 13 is an outer view of the bottom cover.

FIG. 14 shows assembly of the top cover to the bottom cover to provide a housing.

FIG. 15 shows assembly of the wire holding wheel to the tube holding wheel to provide a wheel assembly.

FIGS. 16A and 16B shows connection of the operating wire and the sheathing to the wheel assembly, and relative movement of the operating wheel to the sheathing wheel as controlled by the finger rings to extend or retract the operating wire relative to the sheathing.

FIG. 17 shows the operating wire/sheathing wound onto the endoscopic instrument wheel.

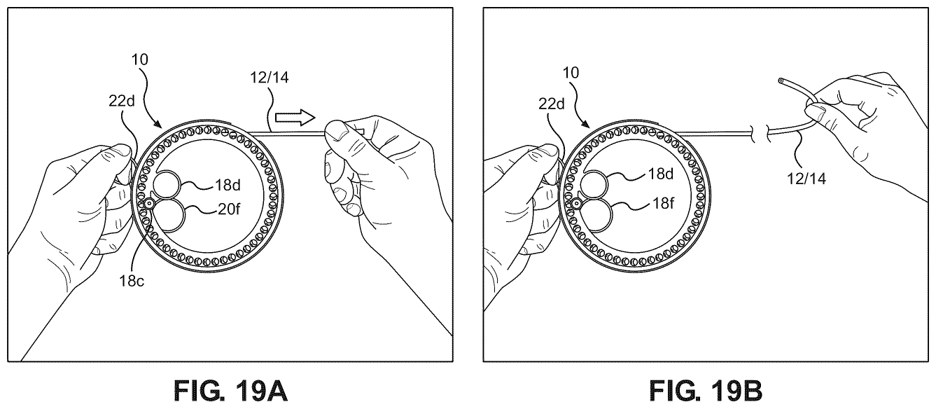

FIG. 18 shows a cross-section of FIG. 17.

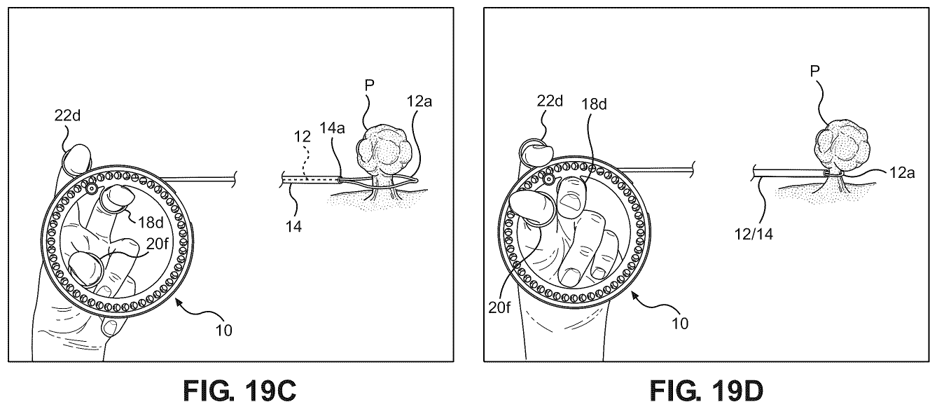

FIGS. 19A-19F depict use of the endoscopic instrument reel.

### DETAILED DESCRIPTION

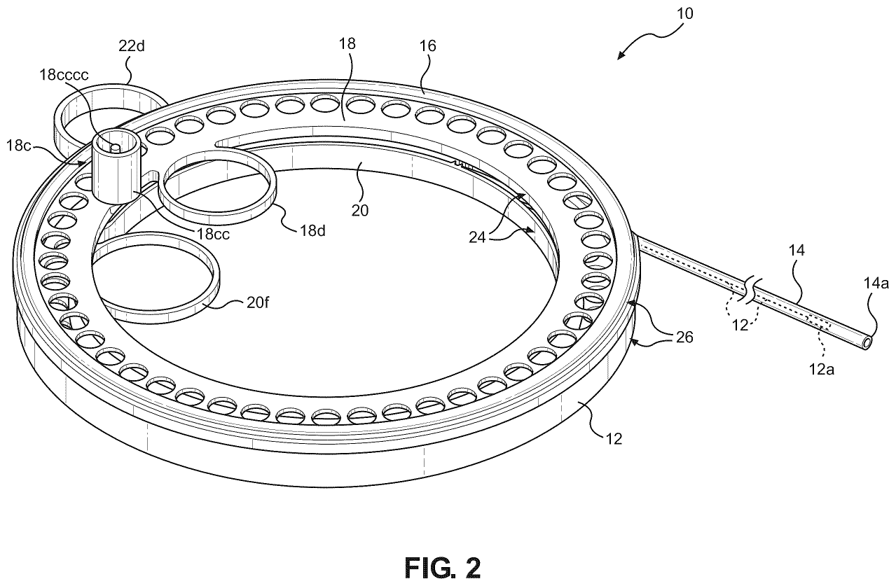

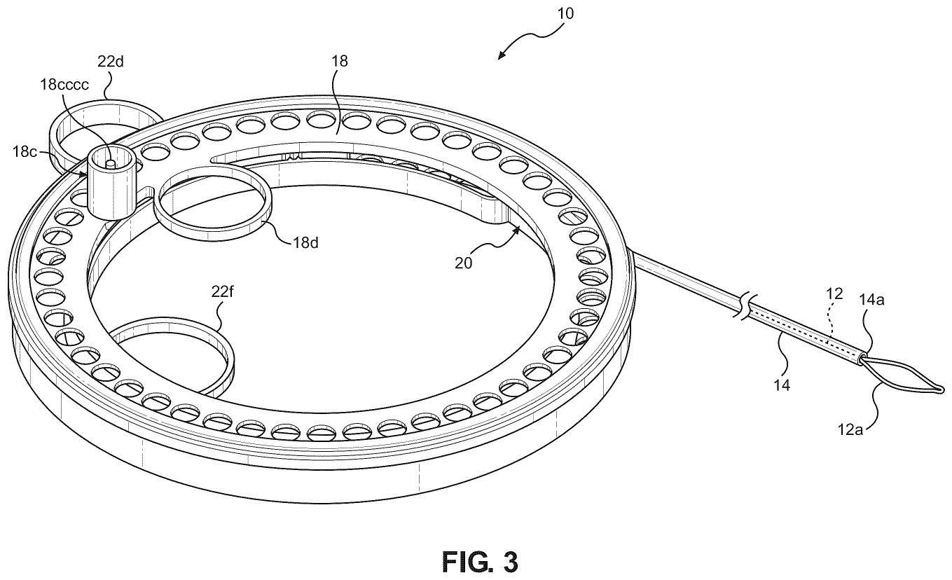

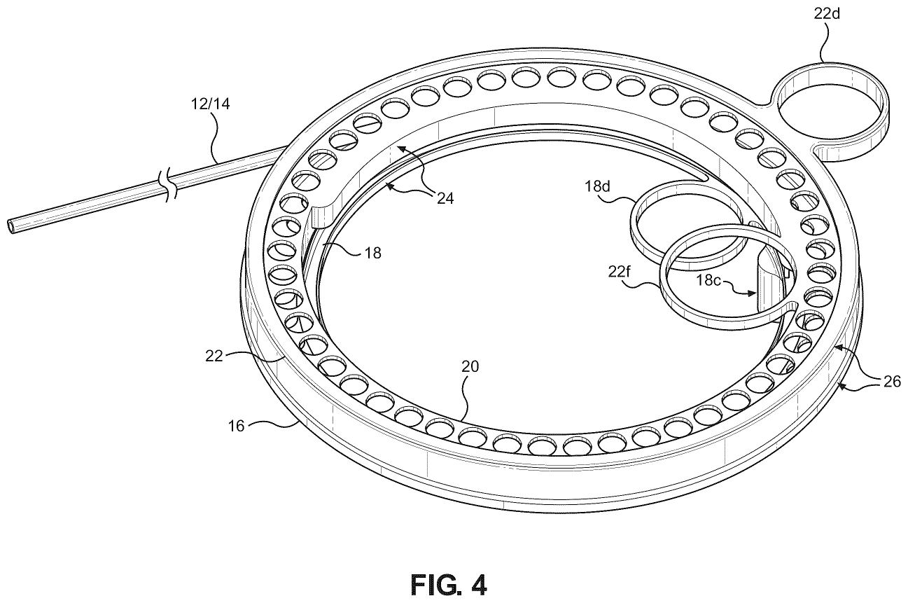

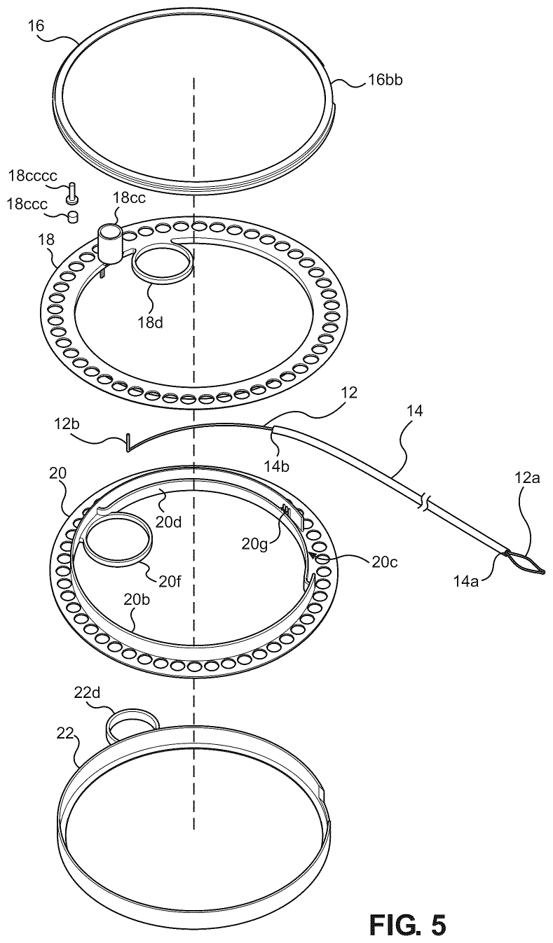

With reference to FIGS. 2-5, the disclosure provides an endoscopic instrument reel 10 of improved construction. The appearance of the endoscopic instrument reel 10 is also configured to be aesthetically pleasing and includes various ornamental aspects and features.

The endoscopic instrument reel 10 is configured to enable manipulation/rotation of an operating wire 12 relative to sheathing 14, and for enclosing and storing the wire 12 and the sheathing 14 prior to deployment from the body of a patient and after withdrawal of the sheathing/wire from the body of the patient to avoid exposure of the sheathing/wire to the surrounding environment and medical personal. The operating wire and sheath structure generally has a length ranging from about 4 feet to over 15 feet, depending on the application.

The endoscopic instrument reel 10 includes a top cover 16, a wire controlling wheel 18, a sheathing holding wheel 20 and a bottom cover 22. The covers 16 and 22 and the wheels 16 and 18 are all desirably of molded plastic construction. The endoscopic instrument reel 10 may be disposable or reusable after sterilization. The endoscopic instrument reel 10 may be provided sterilized in a sealed bag or packaging and disposed of following a surgical procedure.

To provide the assembled endoscopic instrument reel 10, the wire controlling wheel 16 and the sheathing holding wheel 20 and are interlocked together to provide a wheel assembly 24 to receive the wire/sheathing 12/14. The top cover 16 and the bottom cover 22 are installed over the wheel assembly 24 and welded together to provide a housing 26 to enclose the wheel assembly 24.

In brief overview and explained more fully below, the wheel assembly 24 enables the wheels 18/20 to have limited rotation relative to one another to effect relative movement of the wire 12 relative to the sheathing 14. Generally this is done by rotating the wheel 18 relative to the wheel 20. The housing 26 enables the wheel assembly 24 to fully rotate relative thereto for winding and unwinding the wire/sheathing 12/14 into and out of the housing 16.

The operating wire 12 is conventional flexible wire of the type used with endoscopic procedures, and includes a tool or workpiece 12a at the distal end. As shown, the tool 12a is a snare and is generally referred to herein as the snare 12a or tool 12a. It will be understood that the tool 12a may be a variety of endoscopic tools such as forceps, brushes, needles, retrievers, baskets, and the like. The wire 12 is also configured to have a proximal end 12b thereof configured as a wire connector for connecting the proximal end 12b of the wire 12 to the wire controlling reel 18.

The sheathing 14 is a conventional flexible tubing of the type used with endoscopic procedures to slidably receive the operating wire 12. A distal end of the sheathing is denoted 14a and a proximal end of the sheathing is denoted 14b.

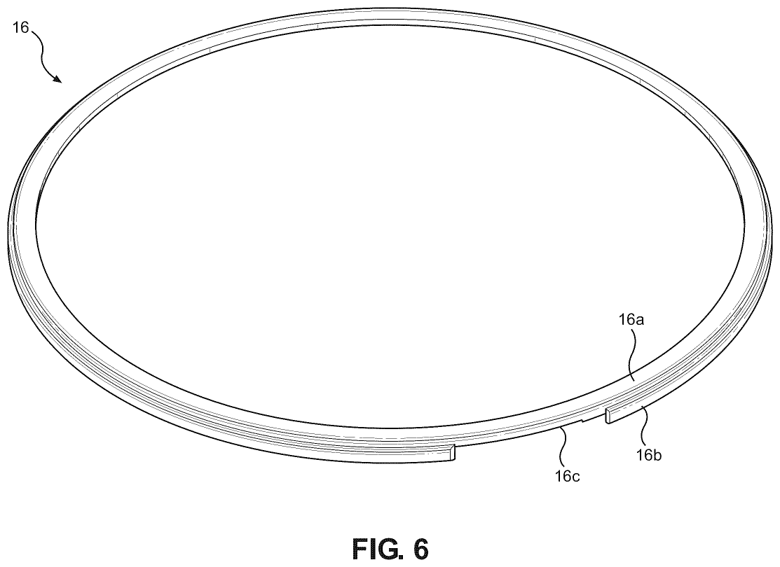

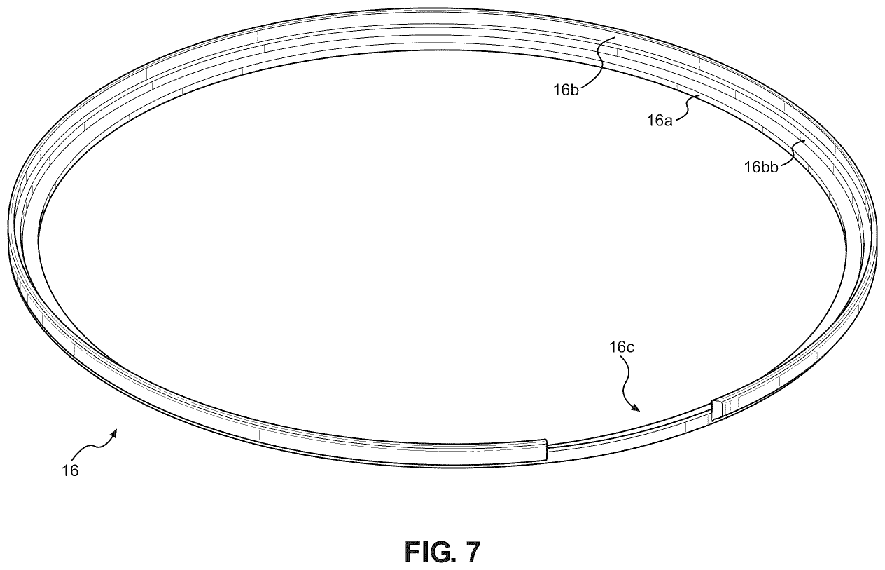

With additional reference to FIGS. 6 and 7, the top cover 16 has a circular upper wall 16a and a depending peripheral sidewall 16b. A gap 16c is provided in the sidewall 16 b for passage of the wire/sheathing 12/14. The interior of the sidewall 16 b has a step 16bb for seating onto the bottom cover 22.

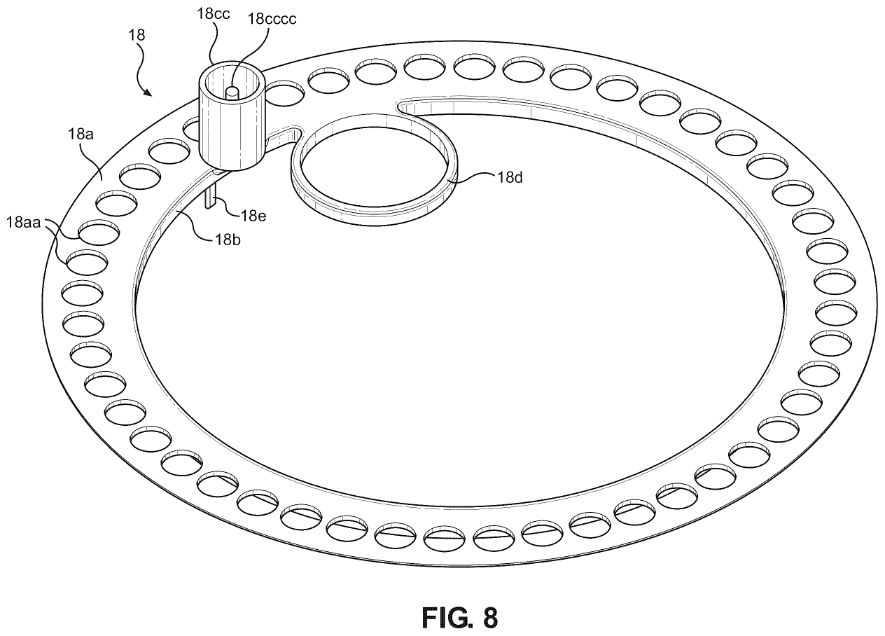

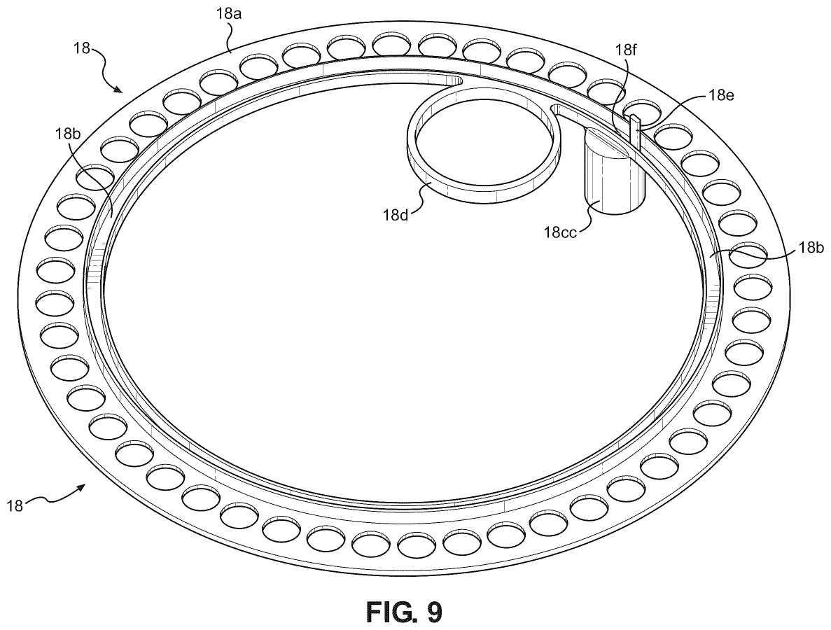

With additional reference to FIGS. 8 and 9, the wire controlling wheel 18 has a circular upper flange 18a. The flange 18a may include one or more apertures 18aa to reduce weight and material usage. As depicted, the apertures 18aa selected and their arrangement also provide an aesthetically pleasing appearance. An inner peripheral channel 18b is defined along the flange 18a.

An electrical connector 18c extends upwardly from the flange 18a and communicates with the channel 18b. The electrical connector 18c is provided for use with endoscopic tools 12a of the type configured to deliver electrosurgical current for providing heat for tissue destruction or coagulation. This is commonly utilized with tools such as forceps and snares. The electrical connector 18c is provided by a plastic bore 18cc as shown, which receives a conductive receiver 18ccc. A lower end of the plastic bore 18ccincludes an aperture for passage of the wire connector 12b of the operating wire 12 to connect to a lower end of the conductive receiver 18ccc seated in the plastic bore 18cc. A plug 18cccc is secured to an upper end of the conductive receiver 18ccc and plugs into or otherwise connects to an electrical cable or other source of incoming electrical current connected to an electrical controller to controllably apply electrical current to the operating wire 12.

A finger ring 18d extends interior of the flange 18a and the channel 18b. A locking pin 18e and a wire receiver 18f are defined in the channel 18b, with the wire receiver 18f in communication with an aperture in the bottom of the plastic bore 18cc. The wire receiver 18f is configured for passage of the wire connector 12b for connecting the proximal end of the wire 12 to the wire controlling reel 18 and to the electrical connector 18c. As shown, the wire receiver 18f is an aperture sized for passage of the wire connector 12b, which is configured as the end of the wire 12 bent at a 90 degree angle. The wire connector 12 b is thereafter securely connected to the conductive receiver 18ccc.

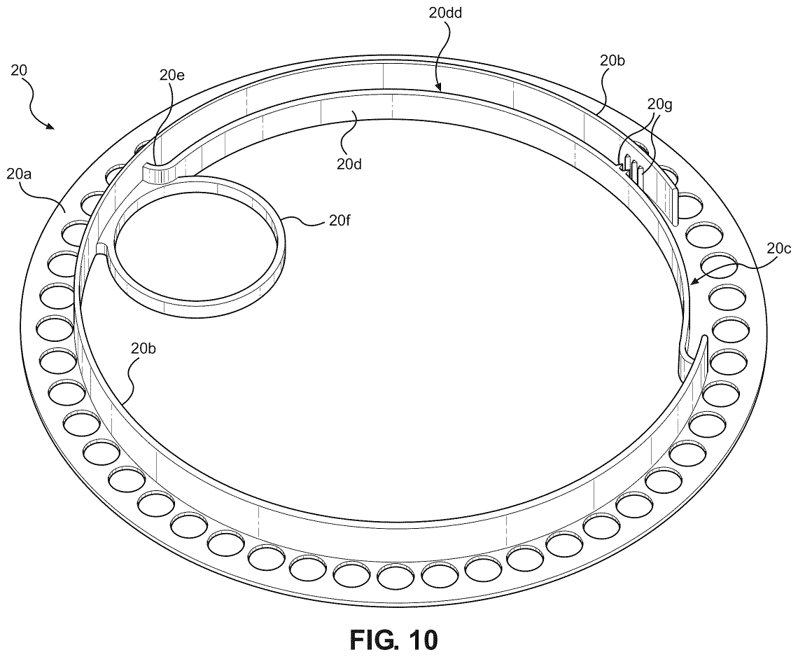

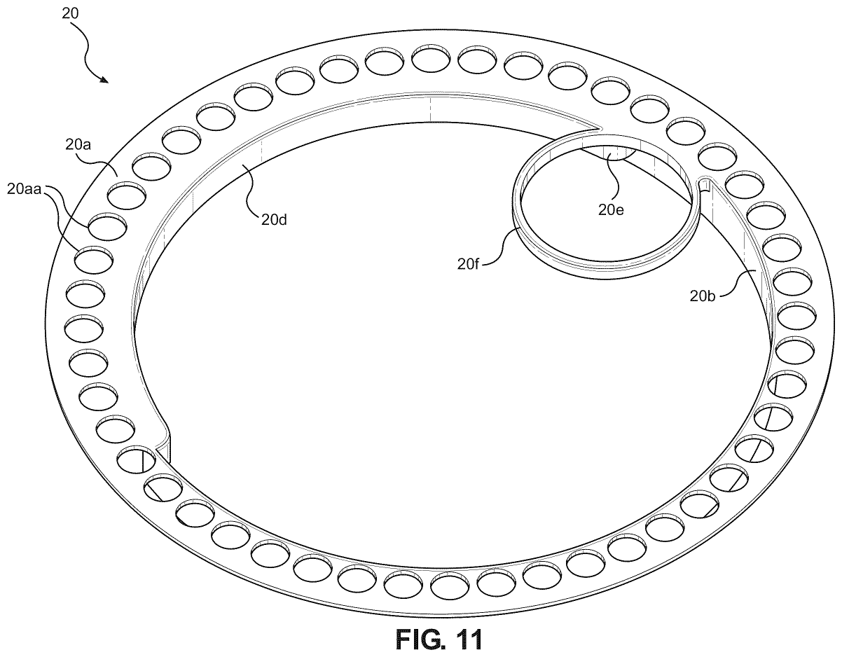

With additional reference to FIGS. 10 and 11, the sheathing holding wheel 20 has a circular lower flange 20a which serves as a channel cover as explained below. The flange 20a may include one or more apertures 20aa to reduce weight and material usage. As depicted, the apertures 20aa selected and their arrangement also provide an aesthetically pleasing appearance.

A sidewall 20b extends upwardly from an inner edge of the flange 20a, except the sidewall 20b is not continuous and defines a gap 20c for cooperating with the gap 16c of the top cover 16 for passage of the wire/sheathing 12/14. A channel wall 20 d extends from an interior edge of the sidewall 20b just before the gap 20c and past the gap 20c to a location on the sidewall 20b past the gap 20c to define a locking channel 20dd that enables rotation of the wire holding reel 18 and the sheathing holding wheel 20 relative to one another.

A stop 20e defined at an end of the locking channel 20dd of the sheathing holding wheel 22 is configured to be engaged by the locking pin 18e of the wire holding wheel 18. Engagement of the locking pin 18e with the stop 20e limits counterclockwise rotation of the wire holding reel 18 relative to the sheathing holding wheel 20 to limit extension of the proximal end 12b of the wire 12 away from the proximal end 14b of the sheathing 14.

A thumb ring 20f extends interior of the flange 20a and sidewall 20b adjacent the distal end of the locking channel 20dd. The locking channel 20dd is sized to receive the sheathing 14 and to secure the proximal end 14b of the sheathing 14 within the locking channel 20dd. The locking channel 20dd includes opposed teeth 20g for engaging the exterior of the sheathing 14. The exterior of the sheathing 14 is preferably sonically welded to the teeth 20g to secure the exterior of the sheathing 14 to the teeth 20g and lock the location of the proximal end 14b of the sheathing 14.

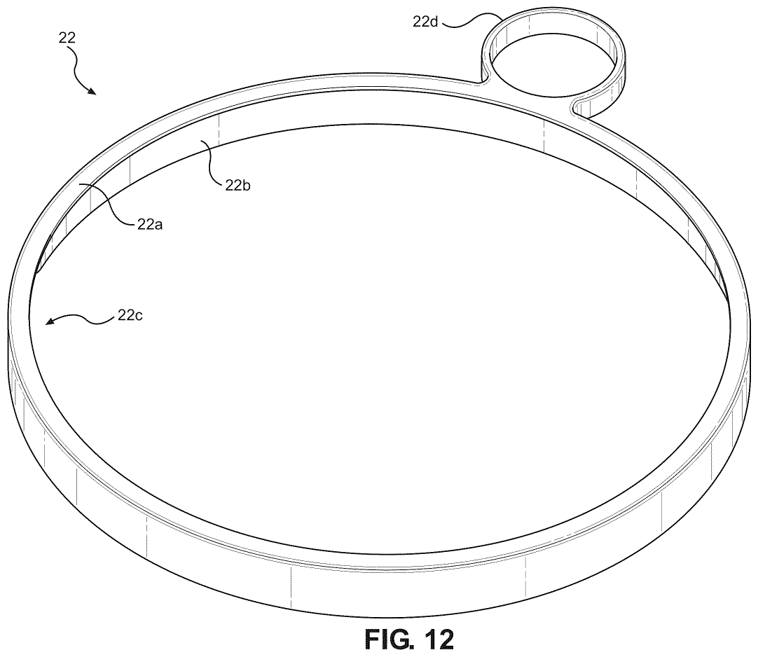

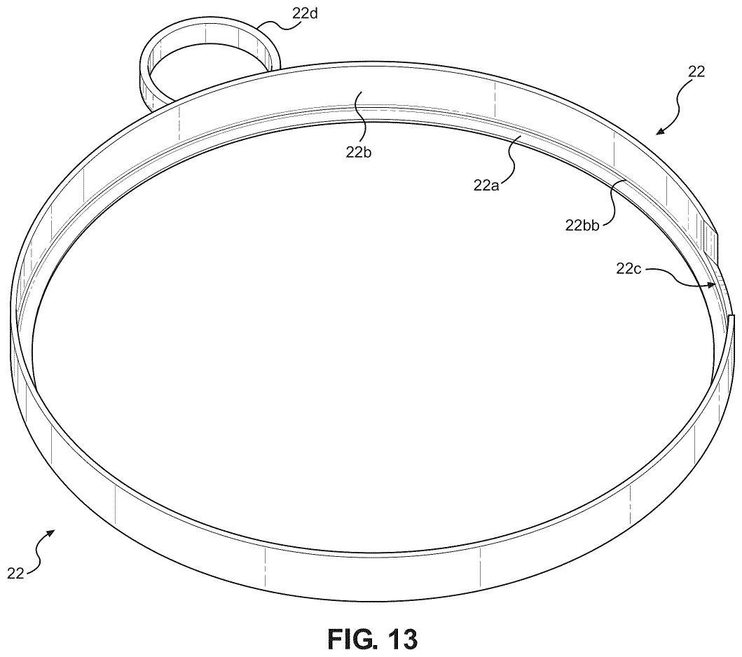

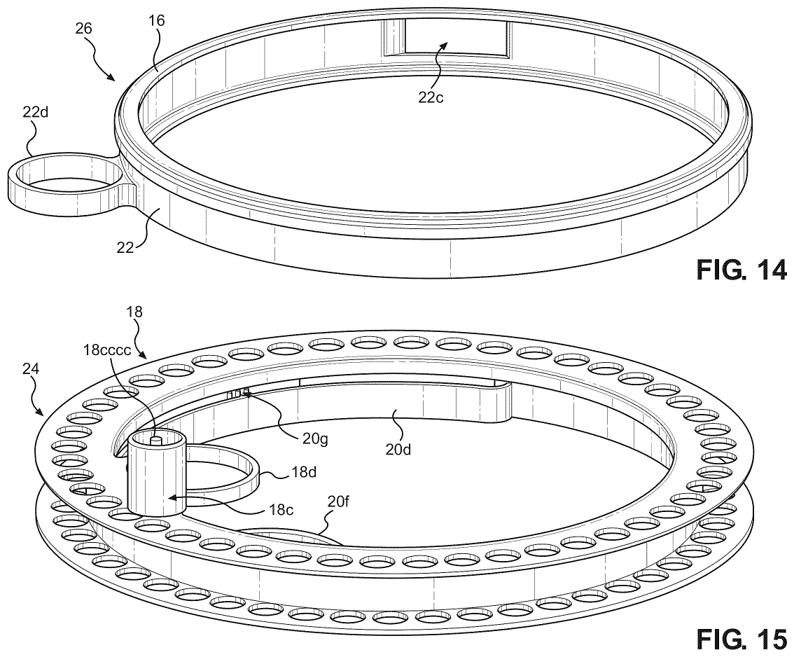

With additional reference to FIGS. 12 and 13, the bottom cover 22 has a circular lower wall 22a and a surrounding peripheral sidewall 22b. A gap 22c is provided in the sidewall 22b for cooperating with the gap 16c of the top cover 16 and the gap 20c of the sheathing holding wheel 22 for passage of the wire/sheathing 12/14. A finger ring 22d extends outwardly from the lower wall 22a and the sidewall 22b. The interior of the sidewall 22b has a rim 22bb for engaging the step 16bb of the top cover 16 when the top cover 16 and the bottom cover 22 are assembled to provide the housing 26 as seen in FIG. 14

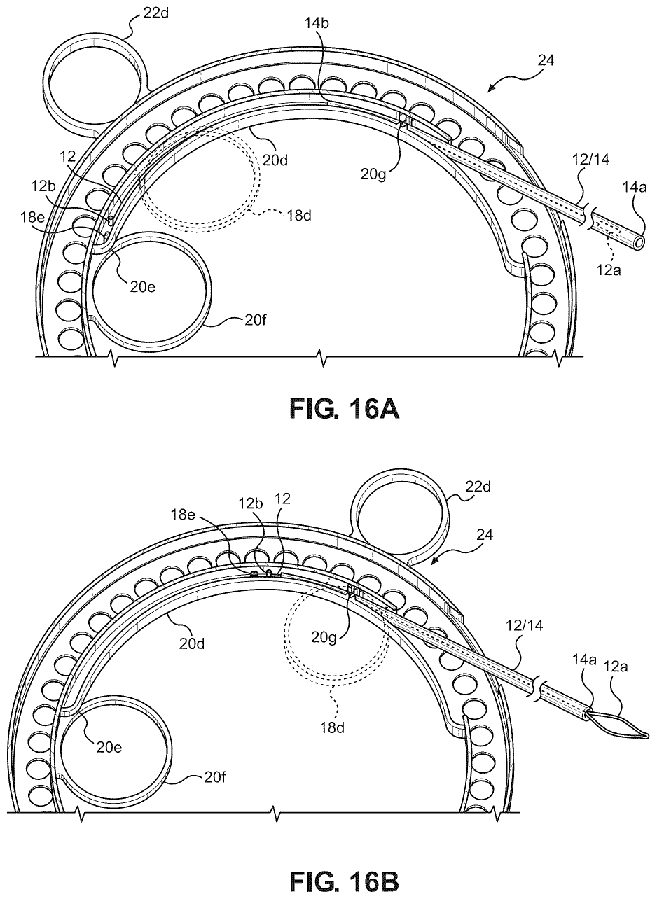

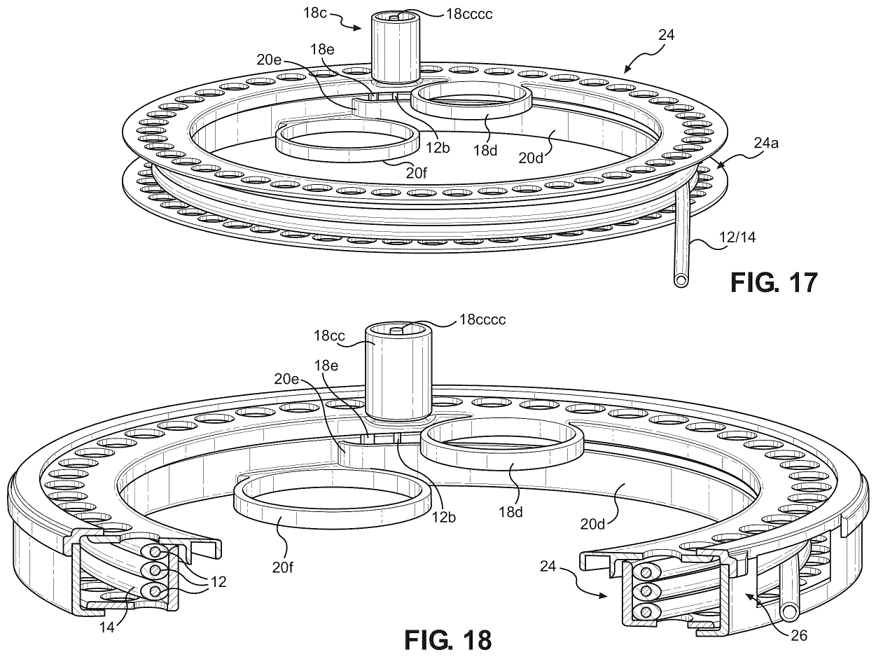

When the wire holding wheel 18 and the sheathing holding wheel 20 are assembled to provide the wheel assembly 24 (FIG. 15), the wire holding wheel 18 and the sheathing holding wheel 20 cooperate to restrict radial movement of the wire holding wheel 18 to a range defined between two limit or end positions. The end positions are boundaries to locate the proximal end 12b of the wire 12 incrementally between a maximum distance away from the proximal end 14b of the sheathing 14 (FIG. 16A) and a minimum distance from, including touching, the proximal end 14b of the sheathing 14 (FIG. 16B). These minimum and maximum distances correspond to the range of motion of the finger ring 18d can have relative to the thumb ring 20f.

The proximal end 12b of the wire 12 travels in the locking channel 20dd corresponding to the range of motion of the finger ring 18d relative to the sheath holding wheel 20. The circular lower flange 20a provides a cover over the locking channel 20dd. The locking pin 18e also slides within the locking channel 20dd as the wire 12 is retracted into the proximal end 14b of the sheathing 14, and as the wire 12 extends away from the proximal end 14b of the sheathing 14 as the finger ring 18d is used to move the wheel 18 relative to the wheel 20. The channel cover provided by the flange 20a protects the user's fingers from contact with the wire 12, which could result in electrical shock and/or damage to components of an endoscope with which the endoscopic instrument reel 10 is utilized.

FIG. 16A shows the wheel assembly 24 when the distal end (tool/snare 12a) of the operating wire 12 is retracted a maximum allowed distance into the distal end 14a of the sheathing 14 with the snare 12a fully closed. The proximal end 12b of the wire 12 is extended away from the proximal end 14b of the sheathing, which distance is the same as the distance that the distal end (tool/snare 12) of the wire 12 is retracted into the distal end 14a of the sheathing 14.

Travel of the proximal end 12b of the wire 12 in the locking channel 20dd in a direction away from the proximal end 14b of the sheathing 14 is limited by the locking pin 18e engaging the stop 20e in the locking channel 20dd. Movement of the proximal end 12b of the wire 12 is accomplished by moving the finger ring 18d to rotate the operating wire wheel 18 relative to the sheathing holding wheel 20. In FIG. 16A, the finger ring 18d is close to the thumb ring 20f.

FIG. 16B shows the wheel assembly 24 when the distal end of the operating wire 12 is fully extended to a maximum allowed distance out of the distal end 14a of the sheathing 14 with the snare 12a fully open. This positions the proximal end 12b of the wire 12 adjacent the proximal end 14b of the sheathing, which distance is the same as the distance that the distal end (tool/snare 12) of the wire 12 is extended past the distal end 14a of the sheathing 14. Travel of the proximal end 12b of the wire 12 in the locking channel 20dd in a direction toward the sheathing 14 is limited by the proximal end 12b of the wire 12, configured as a bend in the wire, engaging the proximal end 14b of the sheathing 14. In FIG. 16B, the finger ring 18d is spaced distant to the thumb ring 20f, the distance therebetween representing a comfortable movement of the finger and thumb utilized by the user in the finger ring 18d and the thumb ring 20f.

The distance that the proximal end 14b of the sheathing 14 extends past the teeth 20g is selected during assembly of the reel 10 and determines the extent of radial movement of the wire 12 in the locking channel 20dd which corresponds to the range of movement of the wire 12 relative to the sheathing 14. That is, the distance that the proximal end 12b extends from the proximal end 14b of the sheathing corresponds to the position of the snare 12a relative to the distal end 14a of the sheathing.

The total range of motion of the wire 12 relative to the sheathing 14 is adjusted by the location of the proximal end 14b of the sheathing 14 from the teeth 20g when the sheathing 14 is secured to the teeth 20g as by sonic welding. The closer the proximal end 14b of the sheathing 14 is to the teeth 20g, the greater the distance that the proximal end 12b of the wire can extend past the proximal end 14b of the sheathing 14.

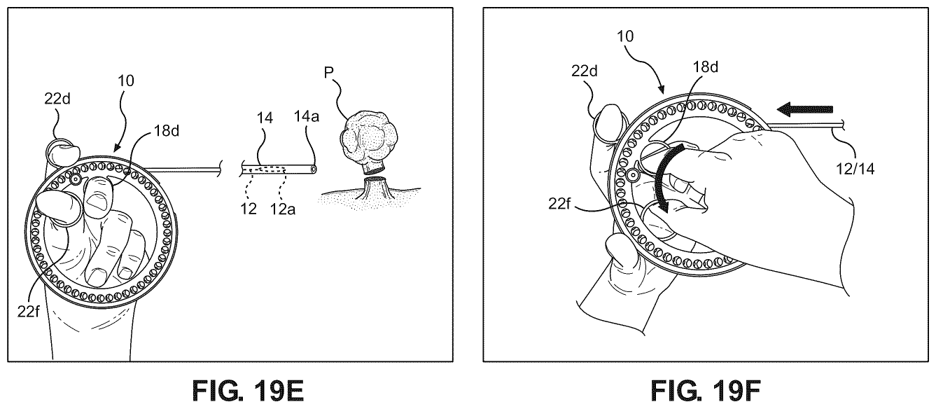

With reference now to FIGS. 19A-19F, there are shown depictions of use of the endoscopic instrument reel 10 from storage, to use as a polypectomy snare, and back to storage. As shown, the snare 12a is being utilized cold, without electrical current, to frictionally cut a polyp P of a type found in the colon of a patient. However, it will be appreciated that the endoscopic instrument reel 10 is configured as described above to connect to electrical power and that such electrical power may be utilized to operate the snare 12a in a hot mode for cutting of the polyp P.

FIG. 19A shows the endoscopic instrument reel 10 prior to use. As will be observed, the wire 12 is retracted into the sheathing 14 and is not visible. The sheathing 14 containing the wire 12 is wound onto the endoscopic instrument reel 10, with just the distal end of the sheathing 14 exterior of the reel 10. Thus, initially for use, the sheathing 14 containing the wire 12 is manually pulled from the reel 10, as depicted in FIG. 19B. To accomplish this, the user holds the reel 10 by the finger ring 22d with one hand, and grasps the distal end of the sheathing 14 with the other hand and pulls the sheathing 14 from the reel 10, rotating the wheel assembly 24 clockwise. The distal end of the sheathing 14 is then introduced into the body of the patient. In this case, entry would be via the rectum of the patient.

As will be noted, the wire 12 is retracted into the distal end of the sheathing 14 the maximum amount, which corresponds to the finger ring 18d and the thumb ring 20f being adjacent one another with the locking pin 18e contacting the stop 20e.

FIGS. 19C-19E depict use of the endoscopic instrument reel 10 to utilize the snare 12a to cut the polyp P. A preferred manner of holding the endoscopic instrument reel 10 for this procedure is shown, with the thumb of one hand in the thumb ring 20f, the index finger of the same hand in the finger ring 22d, and the middle finger of the same had in the finger ring 18f.

As shown in FIG. 19C, the snare 12a is initially positioned over the polyp P to surround a base of the polyp P. To achieve this, the wire 12 is extended out of the distal end of the sheathing 14 to a maximum amount permitted by operation of the endoscopic instrument reel 10. This is done in one-handed operation by holding the endoscopic instrument reel 10 with the thumb of the user inserted in the thumb ring 20f, and the middle finger in the finger ring 18d, preferably the middle finger, and in the index finger in the finger ring 22f. The user then applies hand force via the fingers and thumb to move the finger ring 18d away from the thumb ring 20f until movement is stopped, which corresponds to the proximal end 12b of the wire 12 abutting the proximal end 14b of the sheathing 14. In this position the snare 12a is fully extended out of the tubing 14, with the thumb located away from the index finger and the middle finger

FIG. 19D shows the snare 12a being closed around the polyp P. To accomplish this the fingers are moved to bring the finger ring 18d back towards the thumb ring 22f.

FIG. 19E shows the snare 12a fully closed to cut the polyp P. As seen, the finger ring 18d has now been moved closer towards the thumb ring 22f.

FIG. 19F shows the snare 12a retracted back into the sheathing 14. As seen, the finger ring 18d has now been moved adjacent the thumb ring 22f, corresponding to the orientation of the wheel assembly 24 as shown in FIG. 16A, with the locking pin 18e engaging the stop 20e in the locking channel 20dd.

FIG. 19F depicts re-winding of the wire/sheathing 12/14 back onto the wheel assembly 24 within the housing 26. This may be accomplished by holding the housing 26 with the opposite (left) hand, and using the thumb and index finger (right hand) or just the index finger to rotate the wheel assembly 24 counterclockwise.

Accordingly, the present device advantageously provides an endoscopic instrument reel that has a thumb ring and finger rings for manipulation/rotation of operating wire relative to sheathing, and integrated into an enclosed reel for storing the sheathing/wire prior to deployment from the body of a patient and after withdrawal of the sheathing/wire from the body of the patient to avoid exposure of the sheathing/wire to the surrounding environment and medical personal.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

1. An endoscopic instrument reel, comprising:
a sheathing containing an operating wire slidably located within the sheathing, the operating wire having a proximal wire end extending from a proximal sheathing end of the sheathing and having a distal wire end that can exit and enter a distal sheathing end of the sheathing;
a wheel assembly having a wire controlling wheel and a sheathing holding wheel assembled together to enable the wire controlling wheel to selectively rotate back and forth a limited amount relative to the sheathing holding wheel, the wheel assembly configured to receive the sheathing containing the operating wire,
the wire controlling wheel having a channel, a first finger ring extending inwardly from the channel, and an operating wire receiver and locking pin located in the channel, and
the sheathing holding wheel having a locking channel configured to cooperate with the channel of the wire controlling wheel and sized to receive the sheathing containing the operating wire and configured to fixedly engage the proximal sheathing end within the locking channel, a thumb ring extending inwardly of the locking channel, and a stop located in the locking channel configured to engage the locking pin of the wire controlling wheel and limit rotation of the wire holding wheel when the wire controlling wheel is rotated in a direction and amount to contact the locking pin with the stop; and
a housing enclosing the wheel assembly and having a passage for the sheathing containing the operating wire, the housing including a second finger ring extending in an outwardly direction on a side of the housing opposite of the first finger ring and the thumb ring;
wherein a user may hold the endoscopic instrument reel with a first finger of the user in the first finger ring, a second finger of the user in the second finger ring, and a thumb of the user in the thumb ring, and the user may wind and unwind the sheathing containing the wire to and from the wheel assembly, and the distal end of the operating wire may be selectively moved in and out of the distal end of the sheathing by selectively rotating the wire holding wheel relative to the sheathing holding wheel by use of the first finger ring, the second finger ring, and the thumb ring.

2. The endoscopic instrument reel of claim 1, wherein the distal end of the operating wire includes a tool.

3. The endoscopic instrument reel of claim 2, wherein the tool comprises a snare.

4. The endoscopic instrument reel of claim 1, wherein the housing comprises a first cover and a second cover that mate together.

5. An endoscopic instrument reel, comprising:
a sheathing containing an operating wire slidably located within the sheathing, the operating wire having a distal wire end that can exit and enter a distal sheathing end of the sheathing;
a wire controlling wheel and a sheathing holding wheel assembled together to receive the sheathing containing the operating wire, the wire controlling wheel having a first finger ring, and the sheathing holding wheel having a thumb ring; and
a housing operatively associated with the wire controlling wheel and the sheathing holding wheel, the housing having a passage for the sheathing containing the operating wire, the housing including a second finger ring;
wherein a user may hold the endoscopic instrument reel with a first finger of the user in the first finger ring, a second finger of the user in the second finger ring, and a thumb of the user in the thumb ring, and the user may wind and unwind the sheathing containing the wire to and from the endoscopic instrument, and the distal end of the operating wire may be selectively moved in and out of the distal end of the sheathing by use of the first finger ring, the second finger ring, and the thumb ring.

6. The endoscopic instrument reel of claim 5, wherein the distal end of the operating wire includes a tool.

7. The endoscopic instrument reel of claim 5, wherein the housing comprises a first cover and a second cover that mate together.
